(19) United States
(12) Patent Application Publication
(10) Pub. No.: US 2012/0101399 A1
(43) Pub. Date: Apr. 26, 2012

(54) Respiratory Monitoring System

(76) Inventor: Barry Henderson, Londonderry (GB)

(21) Appl. No.: 13/332,940

(22) Filed: Dec. 21, 2011

(30) Foreign Application Priority Data
Jun. 25, 2009 (GB) 0910987.7
Feb. 17, 2010 (GB) 1002705.0
Jun. 25, 2010 (EP) PCT/EP2010/003826

(51) Int. Cl. A61B 5/08

(52) U.S. Cl. 600/529

(57) ABSTRACT

A system for determining whether a subject is breathing through his nose or mouth and providing feedback to the user. The feedback may take the form of an alarm signal or an instruction to the user. The system advantageously uses an acoustic sensor placed in the user's suprasternal notch.

### RELATED APPLICATIONS

This application claims the benefit of priority under 35 U.S.C. §365(c) to International Application PCT/EP2010/003826, filed Jun. 25, 2010, which claims priority to Application 0910987.7, filed Jun. 25, 2009 and Application 1002705.0, filed Feb. 17, 2010, incorporated herein by reference in entirety.

### FIELD OF THE INVENTION

The present invention relates to respiratory monitoring systems.

### BACKGROUND TO THE INVENTION

Mouth breathing rather than nose breathing has a detrimental effect on an individual's health and performance in particular their respiratory health and performance.

It would be desirable, therefore, to provide a device that is capable of detecting whether an individual is breathing through his mouth or his nose, and of alerting the individual accordingly, or taking other action.

### SUMMARY OF THE INVENTION

Accordingly, a first aspect of the invention provides a system for monitoring breathing, the system comprising at least one sensor capable of detecting one or more physical characteristics of a user that are associated with breathing and generating at least one corresponding sensor output signal; means for determining from said at least one sensor output signal whether said user is breathing through his nose or his mouth; and means for generating at least one system output signal depending on the detection of nose or mouth breathing.

In preferred embodiment, the system, or device, comprises a head that includes at least one acoustic sensor, the head being adapted to fit in the suprasternal notch (also known as the jugular notch) of a user's body. Advantageously, the head includes an annular cushion laterally surrounding said at least one acoustic sensor. The at least one acoustic sensor is preferably covered by a removable sound transmitting cover. The head is preferably carried by a body, the body being adapted so that at least part of the body engages with the user's chest when the head is located in the suprasternal notch. The body, or at least part of it, is advantageously plastic (non-resiliently deformable).

Said system output signal may comprise, or may be co-operable with, means for notifying said user when mouth breathing is determined. For example, said notifying means may comprise one or more of an audio, visual and/or vibrating alarm signal, or means for generating same. Alternatively, said notifying means may comprise a visual display unit and be arranged to display a corresponding message to said user at least in response to said system output signal. The system output signal may also be generated irrespective of which type of breathing is detected, in which case it may include information identifying the type of breathing that has been determined and/or information identifying one or more characteristics of the detected breathing, for example one or more of: ratio of mouth-to-nose breathing, number of total breaths in a time period (respiration rate), number of nose breaths in a time period and/or number of mouth breaths in a time period. Conveniently, said information can be recorded in suitable data storage means included in the system, and/or transmitted to a remote computer for storage and/or analysis. The system may further include means for displaying said information to the user.

Optionally, the system includes a chest expansion monitor including means for monitoring the user's chest expansion while breathing. The monitoring means is advantageously arranged to determine the rate at which the user's chest is expanding and/or contracting, and/or may be arranged to determine the extent to which the user's chest expands while breathing. The chest expansion monitor may be configured to generate an output signal that is indicative of whether the user is breathing through his nose or his mouth based on one or both of these criteria. Alternatively, the chest expansion monitor may generate one or more output signal(s) that is indicative of the determined rate at which the user's chest is expanding and/or contracting, and/or the determined extent to which the user's chest expands while breathing. The chest expansion monitor may comprise one or more of said at least one sensors, especially one or more stress and/or strain sensors for monitoring chest expansion.

Means for attaching the chest expansion monitor to the user's body are preferably provided, for example in the form of adhesive pad(s)/strip(s), one or more straps, a harness and/or a garment.

The system, or device, may include at least one acoustic sensor for distinguishing between mouth and nose breathing.

The system, or device, may include at least one electrical sensor for distinguishing between mouth and nose breathing. The electrical sensor may comprise one or more electrodes arranged to detect neuron activity that is indicative of mouth breathing and/or nose breathing. The electrical sensor(s) may comprise electrocardiogram (ECG) sensors.

In preferred embodiments, output signals generated by the sensors are communicated to a control unit by a respective communication link, which may be wired or wireless. The control unit determines from the received signal(s) whether mouth breathing or nose breathing is occurring, and/or may be arranged to determine one or more other characteristics of the user's breathing, for example one or more of: ratio of mouth-to-nose breathing, number of total breaths in a time period (respiration rate), number of nose breaths in a time period and/or number of mouth breaths in a time period. The control unit may be arranged to cause the information to be stored and/or displayed to the user.

In preferred embodiment, the system, or device, may be arranged determine a ratio of mouth breathing to nose breathing and to generate an output signal if the determined mouth breathing/nose breathing ratio exceeds a threshold.

The system, or device, may include means for recording one or more determined characteristics of the user's breathing (including when nose breathing occurs and/or when mouth breathing occurs) so that a breathing history may be recorded. The recordal of breathing activity may be performed in addition to or instead of providing notification of breathing activity to the user.

Preferred embodiments of the invention provide a training and monitoring device that encourages and trains users to be aware of their nose breathing activity versus mouth breathing activity and to take action to revert to proper breathing.

Some embodiments include a flexible harness or garment with integral sensors and associated electronics and software that detect breathing activity in real-time and advise end-user of recommended action.

A second aspect of the invention provides a method of monitoring a subject's breathing, the method comprising detecting one or more physical characteristics of a user that are associated with breathing; generating at least one corresponding sensor output signal;

determining from said at least one sensor output signal whether said user is breathing through his nose or his mouth; and generating at least one system output signal depending on the detection of nose or mouth breathing. The preferred method involves placing at least one sensor, preferably an acoustic sensor, at the user's suprasternal notch, analysing the output of said at least one sensor, and determining from said analysis whether the subject is breathing through his nose or his mouth.

Preferred features of the invention are recited in the dependent claims.

Embodiments of the invention have numerous applications including, treating and improving medical conditions associated with poor breathing technique or poor compliance with medication, effects of medication, monitoring level of nasal congestion, maximising the beneficial effects of physical exercise and improving performance in a wide variety of sports activities.

A further aspect of the invention provides a system for monitoring breathing, the system comprising a sensor head, preferably comprising an acoustic sensor, said sensor head being adapted for location in the suprasternal notch of a human user. From another aspect the invention provides a method for monitoring breathing, the method comprising placing a sensor, preferably an acoustic sensor, in the suprasternal notch of a human user.

From another aspect, the invention provides a system for monitoring breathing, the system comprising at least one sensor capable of detecting one or more physical characteristics of a user that are associated with breathing and generating at least one corresponding sensor output signal; means for counting the number of breaths taken in a time period; and means for generating at least one system output signal depending on the number of counted breaths.

Further advantageous aspects of the invention will be apparent to those ordinarily skilled in the art upon review of the following description of preferred embodiments and with reference to the accompanying drawings.

### BRIEF DESCRIPTION OF THE DRAWINGS

Embodiments of the invention are now described by way of example and with reference to the accompanying drawings in which:

FIG. 1 is a schematic view of a respiratory monitoring system embodying the invention; and

FIG. 2A shows a plan view from below of a physical embodiment of the respiratory monitoring system;

FIG. 2B shows a side view of the respiratory monitoring system of FIG. 2A; and

FIG. 2C shows a plan view from above of the respiratory monitoring system of FIG. 2A.

### DESCRIPTION OF PREFERRED EMBODIMENT

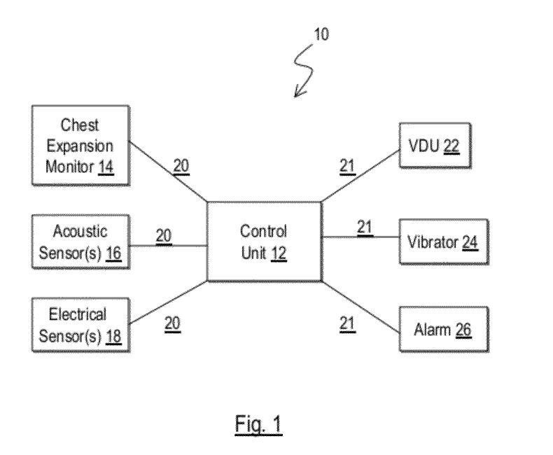

Referring now to FIG. 1, a respiratory monitoring system embodying the invention is shown, generally indicated at 10, and includes a control unit 12, which typically comprises processor, such as a microprocessor or microcontroller. The control unit 12 is programmable, typically by means of suitable computer program code, to operate in the manner described hereinafter.

The system 10 includes at least one, but optionally a plurality of, sensors for monitoring the breathing of a user (not shown). In particular, the, or each, sensor is configured to determine, or to provide an output signal that can be used to determine, whether the user is breathing through his nose or his mouth.

In the illustrated embodiment, the system 10 includes a chest expansion monitor 14, which includes means for monitoring the user's chest expansion while breathing. In particular, the monitoring means is arranged to determine the rate at which the user's chest is expanding and/or contracting, and/or is arranged to determine the extent to which the user's chest expands while breathing. The determined rate at which the user's chest is expanding and/or contracting, and/or the determined extent to which the user's chest expands while breathing are used by the system 10 to distinguish between mouth breathing and nose breathing. For example, mouth breathing typically results in a faster rate of chest expansion and a greater extent of chest expansion than nose breathing. However, the chest expansion monitor may be calibrated in respect of the user's chest expansion characteristics.

The chest expansion monitor 14 may be configured to generate an output signal that is indicative of whether the user is breathing through his nose or his mouth based on one or both of these criteria. Alternatively, the chest expansion monitor may generate one or more output signal(s) that is indicative of the determined rate at which the user's chest is expanding and/or contracting, and/or the determined extent to which the user's chest expands while breathing.

The chest expansion monitor 14 may comprise one or more stress and/or strain sensors (not shown) for monitoring chest expansion.

Means for attaching the chest expansion monitor 14 to the user's body may be provided, for example in the form of adhesive pad(s)/strip(s), one or more straps, a harness and/or a garment. The chest expansion monitor 14 may be incorporated into, or provided on, any such means in any convenient manner. The arrangement is such that, when worn, the chest expansion monitor 14 is suitably located with respect to the user's chest to monitor chest expansion.

Preferred embodiments of the system 10 include at least one acoustic sensor 16, e.g. comprising a microphone or piezo-electric device, for distinguishing between mouth and nose breathing. In one embodiment, one or more first acoustic sensors are provided and arranged to detect nose breathing (e.g. positioned adjacent the user's nose or more particularly the nostril(s)), and one or more second acoustic sensors are provided and arranged to detect mouth breathing (e.g. positioned adjacent the user's mouth). In use, breathing is detected by one or other of the first or second sensors. Alternatively, one or more acoustic sensors are provided and arranged to detect nose breathing and/or mouth breathing and/or to distinguish between the two, or at least to generate an output signal from which the type of breathing can be determined. For example, one or more acoustic sensors may be arranged to detect only one of mouth breathing or nose breathing. In such cases, the absence of detection of said one type of breathing may be taken as an indication that the other type is occurring. Alternatively, one or more acoustic sensors may be arranged to detect whichever of mouth breathing or nose breathing is occurring at a given time. In such cases, the acoustic sensors and/or the control unit may be arranged to analyse the signal produced by the detected breathing to determine its type. This may be achieved by any suitable means, for example comparing the detected signal against a base signal. The base signal may be taken or otherwise established for each user during a calibration process. The analysis of the detected signal may be performed on the time, amplitude and/or frequency characteristics of the signal. In particular, the depth and/or volume of breathing is understood to differ between nose and mouth breathing and these differences may produce respective characteristics in the signal produced by the acoustic sensor(s) that can be used to distinguish between mouth and nose breathing. In any case, the acoustic sensors 16 may be positioned in the vicinity of the user's mouth and/or nose (e.g. on, in and/or adjacent the user's nose and/or mouth). The sensors 16 may be held in place by any suitable means, e.g. one or more straps or adhesive.

The illustrated system 10 includes at least one electrical sensor 18 for distinguishing between mouth and nose breathing. The electrical sensor 18 may comprise one or more electrodes (not shown) arranged to detect neuron activity that is indicative of chest breathing and/or nose breathing. The electrical sensor(s) 18 may comprise electrocardiogram (ECG) sensors.

The output signals generated by the monitor 14 and sensors 16, 18 are communicated to the control unit 12 by a respective communication link 20, which may be wired or wireless. The control unit 12 determines from the received signal(s) whether mouth breathing or nose breathing is occurring. In some cases the control unit 12 is programmed to interpret the output signals (for example by comparing two or more received signals, or otherwise processing the signals) in order to determine whether mouth breathing or nose breathing is occurring, while in other cases, one or more of the received signals provides a direct indication of nose breathing or mouth breathing.

The control unit 12 is arranged to generate one or more output signals depending on whether nose breathing or mouth breathing is detected, and in particular in response to detection of mouth breathing. The output of the control unit 12 may be communicated to one or more output devices (either by wired or wireless links 21), which may include a visual display unit (VDU) 22, a vibrator 24 and/or an alarm unit 26 (which may for example be capable of generating an audio and/or visual alarm). The VDU 22 may be included in a computing device, especially a hand held or wearable computing device such as a PDA, mobile telephone or wrist-worn unit.

Preferred embodiments provide a training and monitoring system that monitors and advises users of their breathing status in particular in relation to their nose breathing versus mouth breathing activity and which can also deliver instructions on action required, or a signal indicating that action is required, i.e. reversion to proper breathing. The system achieves this through the gathering and analysis of combined information from at least one but optionally variety of sensors including stress, acoustic and/or electrocardiogram sensors, each being specifically positioned to differentiate, or to allow differentiation, between oral and nasal breathing.

The preferred system may be used to prevent acute and chronic medical conditions associated with inhalation through the mouth (mouth breathing) in humans or animals, rather than inhalation through the nose, either for extended periods of times, e.g. for patients with sinusitis, rhinitis, asthma, or for short periods of time, e.g. for patients who are hyper-ventilating. The system may monitor and train (by providing feedback via, for example, the VDU, vibrator or alarm) those whose default breathing mechanism is through the mouth rather than the nose.

Although the preferred system is primarily aimed at the improvement of an end-user's breathing technique it may also have applications in measuring and monitoring peak flow rates and advising medication based on such results, for example, the use of an appropriate level of inhaled bronch-dilator. A conventional peak flow detector (not shown) may therefore be provided in the system 10, arranged to measure peak flow of the user's breathing. The measured peak flow data can be presented to the user together with the detected nose breathing vs. mouth breathing data in order that the user may learn how his breathing habits are affecting his peak flow. In addition the device may also advise on the use of cortico-steroid or preventative inhaled medication and/or nasal decongestion.

In one embodiment, the system compromises:
(a) a harness, shirt (or other garment), adhesive array or similar with:
   (i) an integral or an attached array of sensors at specific nodes or positions to detect strain caused by chest wall extension and/or single or an array of electrodes that detect neurone activity indicative of chest breathing and/or nose breathing.
   (ii) an integrated or attached programmable micro-processor unit with associated embedded computer program code capable of processing and feeding back information in real-time to warn of improper breathing technique and producing a signal indicative of the problem (descriptive of abnormal breathing) and optionally providing instructions for corrective action.

The control unit may detect strain across an array of sensors and filter out information relating to false readings, e.g. false positives caused by sensors being knocked or jolted out of position.

The harness, garment or adhesive array may be worn next to the body or over clothes. In use, the system gathers information relating to breathing status through integral or attached sensor technologies. Information that is directly or indirectly indicative of the user's type of breathing is recorded utilising the outputs generated by one or more of the following: stress gauges, acoustic sensors and electrocardiogram sensors positioned at specific anatomical nodes for optimal detection and analysis. Collection of this data will allow subsequent analysis to be performed which will investigate both the temporal and spectral characteristics of the relevant signals produced by the system 10, which will aim to offer the discriminatory information required.

The preferred system is capable of multi-modal signal and feedback mechanisms.

Feedback to the user may be audio including voice and/or visual and/or vibration. The method of feedback may involve a separate device representing the end-user interface (e.g. including the VDU 22). The transfer of information within the system may be wireless e.g. Radio Frequency (RF) or Bluetooth or via a connecting lead or similar e.g. USB to phone, PDA, computer or other device (any of which may include the VDU 22). Feedback to the user may be through transmission to a wrist worn unit or a pager device (either of which my replace or incorporate the VDU 22 described above) thereby increasing comfort and acceptability to the user.

As well as providing feedback on breathing trends and suggested remedial action the system may provide feedback aimed at optimising user's benefit from intra nasal nitric oxide release. For example, excessive mouth breathing can result in the user being deprived of desirable levels of nasal nitric oxide release. Therefore, in response to detecting excessive mouth breathing, the system 10 may cause a message to be issued to the user which, in this example, might be a remedial instruction such as “exhale slowly through the nose for X seconds”, or “inhale through nose for X seconds”. Again such a feedback may be audio including voice and/or visual and/or vibration. The preferred system is capable of identifying differences in patterns between nasal and mouth breathing and hence integrate this information/knowledge into a decision support system which will form the basis of the patient monitoring and motivational feedback to recommend the change from mouth to nasal breathing.

The preferred system is programmable to reflect the user's own activity status, e.g. resting, at work, sports, sleeping, and/or to reflect the user's own disease state, e.g. well controlled asthma, poorly controlled asthma, mild asthma, COPD, Cystic Fibrosis or sleep apnoea. The feedback provided by the system may then depend on the programmed state of the user. The system may also include an input/output function enabling calibration for each user, e.g. chest extension due to mouth breathing versus chest extension due to nose breathing.

The preferred system records and differentiates between mouth and nose breathing in real time. Mouth breathing typically results in both a greater and faster rate of extension of the chest wall compared with nose breathing.

The system may be arranged to give a signal if the mouth breathing/nose breathing ratio exceeds a certain threshold. This ratio may be recorded over time intervals, e.g. every 10 minutes, so that an accurate record is kept during the course of the day.

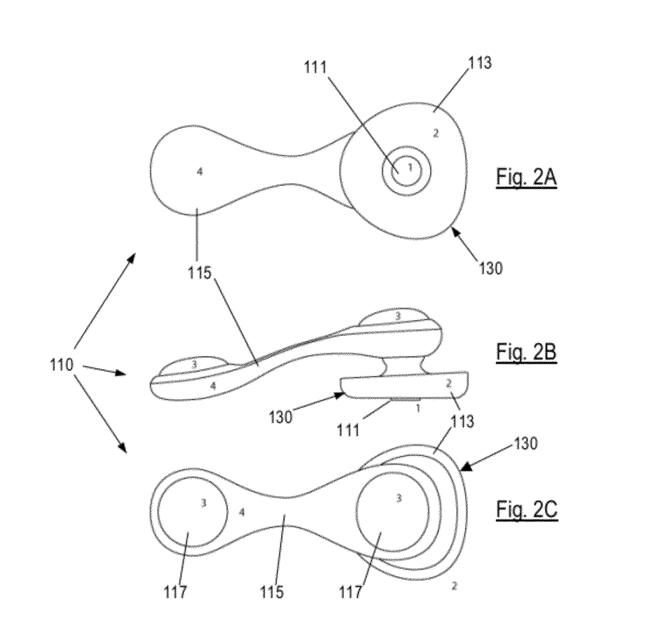

Referring now to FIG. 2, a preferred embodiment of the invention is described. The device shown in FIG. 2 may be used with, or provided with, any one of or any combination of the features of the system described with respect to FIG. 1. FIG. 2 shows a respiratory monitoring system comprising a device 110 having a monitoring head 130 that is provided with a sensor (not visible), preferably an acoustic sensor (e.g. a microphone or piezo-electric acoustic sensor). The sensor is preferably covered in use by a removable sound transmitting cover 111, which may for example be made from plastics. In preferred embodiments, a single acoustic sensor is provided in the head, although in alternative embodiments more than one acoustic sensor may be provided. In alternative embodiments, more than one sensor may be provided in the head 130, the or each sensor may be an acoustic sensor, or an alternative form of sensor, or a combination of more than one type of sensor.

It has been found that the suprasternal notch, also known as the jugular notch, of a human body is a particularly suitable location for monitoring acoustic signals that are generated by breathing. Accordingly, the head 130 is shaped and dimensioned to fit in the suprasternal notch. The shape and dimensions of head 130 are advantageously such that they facilitate retention, and preferably self-retention, of the head 130 in the suprasternal notch. The preferred embodiment employs a single acoustic sensor located, in use, at a single anatomical point on the user's body.

Preferably, at least part of, for example a peripheral part of, the head 130 is deformable to facilitate fitting the head to the suprasternal notch. In the preferred embodiment, the head 130 includes a peripheral cushion 113, which is preferably formed from a deformable material, e.g. a soft polymer. The cushion 113 facilitates creating a close fit between the head 130 and the user's body. This aids transmission of acoustic signals from the body to the sensor and minimises external noise interference. The cushion 113 is typically annular in shape such that it laterally surrounds the acoustic sensor and, when present, the cover 111.

The device 110 preferably includes means for retaining the head 130 on the user's body, and in particular in the user's suprasternal notch. The retaining means can take any suitable form, for example a strap, harness and/or adhesive. In the preferred embodiment, however, the retaining means is provided by a body 115 that carries the head 130. The body 115 extends from the head 130 and is shaped and dimensioned to engage with the user's chest when the head 130 is located in the suprasternal notch. To this end, the body 115 may be wholly or partly formed from a material that is capable of being deformed and retaining its deformed shape, e.g. a flexible polymer, or any other suitable self-supporting non-resilient deformable material (fictile or plastically deformable). Typically, the head 130 is located at one end of the body 115, a chest contact point being located at the other end. Together, the head and the body engage with the user's body to retain the device in place at least when the user is at rest. Optionally, one or more adhesive patches may be provided on the head and/or body, and/or one or more straps may be provided, to help keep the device 110 in place.

The sensor is capable of detecting acoustic signals that are generated by the user's body when breathing. The characteristics of such acoustic signals differ depending on whether the user is breathing through his nose or his mouth. The device 110 preferably includes, but may otherwise be co-operable with, means for determining from the output of the sensor whether the user is breathing through his nose or his mouth. The capability of differentiation between mouth and nose breathing may for example be achieved empirically by comparison with reference data obtained, for example, during a calibration process.

The determining means is conveniently provided by a control unit such as the one described above in relation to FIG. 1. The control unit may be provided on the device, for example in the head 130, or in/on the body 4, or may be provided remotely from the device 110. The control unit may record data derived from the sensor output and/or may be co-operable with means for generating an alarm. The device 110 may also include a wireless transmitter (not shown) for communicating data gathered and/or computed by the device 110 to a remote computer. In the case where the device does not have an on board control unit for analysing the detected acoustic signals, the sensor output can be transmitted to a remote location for analysis. The device 110 may include an alarm unit (not shown), for example a device for generating an audio, visual and/or vibration alarm. The device 110 may include a display unit (not shown) for displaying data gathered and/or computed by the device 110 to the user, and/or a message relating to same. The device 110 may include an audio unit (not shown), for example including a speaker, for rendering data gathered and/or computed by the device 110 to the user and/or a message relating to same. The device 110 may include a data storage device for storing data gathered and/or computed by the device 110. Alternatively, one or more of the alarm unit, display unit, audio rendering unit and storage device may be provided remotely from the device 110, the device 110 including means, e.g. a wired or wireless communications link, for communicating with same.

It will be seen that the preferred device is capable of determining between mouth and nasal breathing, using a single acoustic sensor at a single anatomical point—the suprasternal notch.

The device may include one or more control surfaces 117, e.g. buttons, to allow user control of the device.

The acoustic sensor device may for example comprise a piezoelectric acoustic sensor, e.g. a thin film piezoelectric acoustic sensor. Conveniently, the sensor is of a type comprising a substrate having a layer of piezoelectric film disposed on a curved portion of the substrate second surface and a layer of flexible material disposed on a second surface of the piezoelectric film opposite the first surface for contacting with an object to be sensed. This electronic stethoscope operates as a vibration sensing element contacted to a body part such as neck or throat so as to detect sound propagating through tissue, muscle, tendon, ligament and bone. In this way an acoustic signal is detected by the sensor.

As an alternative to placing the acoustic sensor at the suprasternal notch (or more particularly on the hollow point directly above suprasternal notch), one or more acoustic sensors could be placed directly under the chin, on the windpipe, and/or on the neck, especially the right and/or left hand side of the neck. It is found however that placing the acoustic sensor(s) in the suprasternal notch gives more reliable results.

The determining means is conveniently implemented by one or more computer program supported by the control unit 12. One or more conventional signal processing methods may be used to determine whether the output signal of the acoustic sensor is indicative of mouth breathing or nasal breathing, including Short Time Fourier Transform, Filter Analysis, Linear Predictive Coding (LPC) Analysis and/or Wavelet Analysis. Typically, the method involves extraction of features from the signal to make the determination. The determining means may be calibrated before use, for example using calibration data from the end user or from one or more test users. In either case, the calibration data is gathered by the use(s) breathing through their mouth for a period, and breathing through their nose for a period. By analysis of the resulting output signals, the control unit can learn to distinguish between the two. In one embodiment, the determining means includes or is operatively associated with an artificial neural network, especially a Back-propagation Neural Network, that is trained on the calibration data and arranged to distinguish between nose and mouth breathing during use.

In subjects with healthy lungs, the frequency range of the vesicular breathing sounds typically extends to 1000 Hz, whereas the majority of the power within this range is found between approximately 60 Hz and 600 Hz. Other sounds, such as wheezing or stridor, can sometimes appear at frequencies above 2,000. In preferred embodiments, the device 10, 110 is arranged to analyse output signals in the range 1 Hz to 1000 Hz range, and in particular frequencies below 600 Hz. It is particularly advantageous to analyse frequencies below 100 Hz, or approximately 100 Hz, since it is found that signal features relating to lung sound can most readily be detected in that range. Different frequency bands may be used for different functions, for example the frequencies under 100 Hz may be isolated to extract acoustic features, higher frequencies above 1200 Hz carry a lot of noise which may be filtered out at an initial stage, while the frequencies in between may be used for end-point detection (i.e. detecting the beginning as well as the ending of a meaningful sound signal). So the original signal may pass through several low pass and/or band pass filters to cut out the specified frequency bands.

End point detection, which is associated with feature extraction, may be performed in the time domain, using conventional techniques such as short-time average amplitude, short-time average zero crossing rate and short-time energy.

The determination of mouth or nose breathing may be performed in a plurality of states. Firstly, audio files that contain the breathing sound are recorded by the acoustic sensor(s). Alternatively the processing of the sensor output may be performed in real time. The next stage involves signal pre-processing, which takes place before feature extraction and may involve filtering out certain frequency bands, e.g. signal noise, framing and windowing the signal and performing a Fast Fourier Transform. Characteristic or feature extraction can then take place and typically involves End-point Detection and band-pass filtering. The results of the feature extraction may then be provided to the artificial neural network. For the recognition stage, the test data is input to the neural network for detection using the weights obtained in the training process, and some expert experience may be added manually to the result of the detection.

Devices embodying the invention may be used in a variety of applications including, but not limited to, treatment of respiratory conditions such as asthma, sinusitis, rhinitis, sleep apnoea, chronic obstructive pulmonary disease (COPD), cold and flu and wider healthcare applications in areas such as diabetes, hypertension, stroke, cardiovascular disease. They may also be used to assess medication compliance and effectiveness, e.g. in clinical trials.

Optionally, embodiments of the invention are arranged to count the rate at which the user is breathing, e.g. in terms of number of breaths in a given period. This may include a count of all breaths irrespective of whether they are mouth or nasal, and/or may count mouth breaths and/or nose breaths separately. An alarm or other message can be generated if the determined rate or count fails to meet a threshold, and/or the data can be recorded for later analysis and/or rendered to the user (e.g. via a VDU). Knowledge of breathing rate can be useful in sporting applications as well as medical applications.

Optionally, embodiments of the invention are arranged to determine the ratio of nose breathing to mouth breathing. An alarm or other message can be generated if the determined ratio fails to meet a threshold, and/or the data can be recorded for later analysis and/or rendered to the user (e.g. via a VDU). Knowledge of this ration can be helpful in optimising sporting performance as well as optimising breathing for health reasons.

Although embodiments of the invention are particularly suitable for use with human subjects, they could also be used with animals, especially animals having a suprasternal notch or similar anatomical feature.

The invention is not limited to the embodiments described herein, which may be modified or varied without departing from the scope of the invention.

1. A system for monitoring breathing, the system comprising at least one sensor capable of detecting one or more physical characteristics of a user that are associated with breathing and generating at least one corresponding sensor output signal; means for determining from said at least one sensor output signal whether said user is breathing through his nose or his mouth; and means for generating at least one system output signal depending on the detection of nose or mouth breathing.

2. A system as claimed in claim 1, wherein said system comprises a sensor head incorporating an acoustic sensor, said sensor head being adapted for location in the suprasternal notch of a user.

3. A system as claimed in claim 2, wherein said sensor head is incorporated into a device adapted to be fitted to the user's body, the device further including means for retaining itself on the user's body.

4. A system as claimed in claim 3, wherein said retaining means comprises a body extending from said head, the body being adapted so at least part of the body engages with the user's chest when the head is located in the suprasternal notch.

5. A system as claimed in claim 4, wherein said body is adapted such that a free end of the body, distal said sensor head, is engagable with a location on the user's chest below the suprasternal notch.

6. A system as claimed in claim 4, wherein at least part of the body is formed from a plastically deformable material.

7. A system as claimed in claim 3, wherein the head includes an annular cushion laterally surrounding said at least one acoustic sensor.

8. A system as claimed in claim 3, wherein said at least one acoustic sensor is preferably covered by a removable sound transmitting cover.

9. A system as claimed in claim 1, wherein said means for generating at least one system output signal is arranged to generate an output signal at least in response to determining that said user is breathing through his mouth.

10. A system as claimed in claim 1, further including means for determining a ratio of detected mouth breathing to detected nose breathing.

11. A system as claimed in claim 10, wherein said means for generating at least one system output signal is arranged to generate an output signal in response to determining that said ratio fails to satisfy a threshold value.

12. A system as claimed in claim 1, further including means for counting the number of breaths taken by the user in a time period.

13. A system as claimed in claim 12, wherein said means for generating at least one system output signal is arranged to generate an output signal that is indicative of the counted number of breaths, and/or in response to determining that the counted number of breaths fails to satisfy a threshold value.

14. A system as claimed in claim 12, wherein said counting means is arranged to count only the number of mouth breaths or the number of nasal breaths.

15. A system as claimed in claim 1, wherein said means for generating at least one system output signal is arranged to generate an output signal that is indicative of whether mouth breathing or nose breathing is detected.

16. A system as claimed in claim 1, further including means, responsive to said system output signal, for communicating a corresponding message to a user.

17. A system as claimed in claim 16, wherein said message comprises an alarm, said system further including means for generating an alarm.

18. A system as claimed in claim 17, wherein said alarm generating means comprises means for generating one or more of an audio, visual and/or vibratory alarm.

19. A system as claimed in claim 16, wherein said message comprises an instruction, said system further including means for rendering an instruction to the user.

20. A system as claimed in claim 19, wherein said rendering means comprises a visual display unit and/or an audio speaker.

21. A system as claimed in claim 1, wherein sad at least one sensor comprises one or more acoustic sensor, said determining means being arranged to distinguish between mouth or nasal breathing by analysing one or more characteristics of the output of said one or more acoustic sensors, for example the amplitude, temporal and/or frequency characteristics.

22. A system as claimed in claim 1, wherein said determining means comprises a chest expansion monitor including means for monitoring the user's chest expansion while breathing.

23. A system as claimed in claim 1, wherein said determining means comprises at least one electrical sensor for distinguishing between mouth and nose breathing, for example electrocardiogram (ECG) sensors.

24. A system as claimed in claim 1, further including means for storing data representing one or more characteristics of the user's breathing determined from said system output signal.

25. A system as claimed in claim 24, wherein said characteristics include one or more of:
when nose breathing occurs, when mouth breathing occurs, ratio of mouth-to-nose breathing, number of total breaths in a time period (respiration rate), number of nose breaths in a time period and/or number of mouth breaths in a time period.

26. A system as claimed in claim 24, further including means for rendering said characteristics data to a user.

27. A method of monitoring a subject's breathing, the method comprising detecting one or more physical characteristics of a user that are associated with breathing; generating at least one corresponding sensor output signal; determining from said at least one sensor output signal whether said user is breathing through his nose or his mouth; and generating at least one system output signal depending on the detection of nose or mouth breathing.

28. A method as claimed in claim 27, further including placing an acoustic sensor at the user's suprasternal notch, analysing the output of said sensor, and determining from said analysis whether the subject is breathing through his nose or his mouth.
